(19) United States
(12) Patent Application Publication
(10) Pub. No.: US 2005/0102167 A1
(43) Pub. Date: May 12, 2005

(54) Provisioning and controlling medical instruments using wireless data communication

(76) Inventor: Ashok K. Kapoor, San Mateo, CA (US)

Correspondence Address: Ray K. Shahani, Esq.;ATTORNEY AT LAW, Twin Oaks Office Plaza, 477 Ninth Avenue, Suite 112, San Mateo, CA, 94402-1854, US

(21) Appl. No.: 10/957,169

(22) Filed: Sep. 30, 2004

Related U.S. Application Data
(60) Provisional application No. 60/518,637, filed on Nov. 12, 2003.

(51) Int. Cl. A61B 5/02; G06F 17/60

(52) U.S. Cl. 705/3

(57) ABSTRACT

This invention teaches a method of automating some of the tasks requiring continuous data collection at the patient bedside in a hospital in a manner which significantly reduces the chances of error in providing treatment. These tasks include provisioning of the IV pumps or other fluid infusion pumps, feed pumps, oxygen delivery systems, gathering, recording, storing, and analyzing signals from ECG machine or pulse oxymeter or any other medical device. This invention teaches the use of wireless transceiver modules which are connected to the data ports on the medical instrument to gather the data and transmit the data to a wireless access point. Protocols to identify the patient, care provider, medicine, equipment, and treatment are described. Use of an external means for verifying the identity of the medical device and the medicine is also described.

### RELATED APPLICATIONS

This Application is related to and incorporates herein by reference in its entirety U.S. Provisional Application Ser. No. 60/518,637 filed Nov. 12, 2003 entitled METHOD OF PROVISIONING AND CONTROLLING IV PUMPS AND OTHER MEDICAL INSTRUMENTS USING WIRELESS DATA COMMUNICATION, and claims any and all benefits to which it is entitled therefrom.

### FIELD OF THE INVENTION

This invention relates to a method for arranging communication between multiple medical and non-medical devices at the bedside of a patient, where each device is equipped with an apparatus for electronic data communicating using standards protocols such as serial data communication protocol (RS232, RS422, Ethernet, USB, firewire and other similar protocols), parallel data communication protocols, or any other manufacturer specific protocol to allow the users to exchange information with these devices over with a data network.

### BACKGROUND OF THE INVENTION

In any given medical treatment, numerous tasks require continuous data collection at the patient bedside in a hospital in a manner in order to significantly reduce the chances of error in providing treatment. These tasks include provisioning of the IV pumps or other fluid infusion pumps, feed pumps, oxygen delivery systems, gathering, recording, storing, and analyzing signals from ECG machine or pulse oxymeter or any other medical device.

As an example, injecting medicine intravenously is a commonly used method for delivering medication to patients. This task is performed by using a pump to deliver a controlled amount of medicine in a certain time to the patient by using a pump to control the flow of the medicine. Existing intravenous or IV pumps at the patient bedside are manually controlled. The drug flow through the pump is controlled by using a flow control mechanism on the pump which is controlled electronically. The pump also contains associated electronics to display the rate of medicine delivery and other relevant pump parameters on the display panel. The process of setting the flow rate of medicine typically requires manual intervention. It is performed by a nurse, following a physician's instructions. The work flow of a nurse for setting up an IV at the patient bedside consists of rolling the IV pump at the bedside, hanging the appropriate medicinal solutions contained in pouches from the IV pump, and connecting the output of the pouches through the IV pump to the patient. The IV is started by a nurse and at some point, the record is made by the nurse in patient charts of the medicine given to the patient. The details of the flow rate of the medicine are often not recorded, or done only at the beginning of the process. Any changes at a later time in the status of the IV may not be recorded. The process of identifying the time to change the bag is purely by conjecture, as a nurse repeatedly looks at the fluid remaining in the pouch and makes her own judgment regarding the time when a change would be needed, and comes back to make the appropriate changes. This puts a significant burden on the nurses to determine the time for changing medicine delivery apparatus on the pump, as they have to walk over to the patient bedside to determine the status of the pump.

### ADVANTAGES AND SUMMARY OF THE INVENTION

This invention teaches a method for arranging communication between multiple medical and non-medical devices at the bedside of a patient, where each device is equipped with an apparatus for electronic data communicating using standards protocols such as serial data communication protocol (RS232, RS422, Ethernet, USB, firewire and other similar protocols), parallel data communication protocols, or any other manufacturer specific protocol to allow the users to exchange information with these devices over hard wired connections. One embodiment of this invention teaches the use of hardware for electronic data exchange with these devices from hard wired connections to a wireless connection with uniform data format compliant with a formal protocol named Sensitron Personalization Protocol or “SPP”, as described in U.S. patent application Ser. No. 10/850,527 completely assigned to the Applicant herein having filing date May 19, 2004. This method of wireless data communications from the medical device includes the use of any of the existing technologies such as radio frequency transmission, infra red communication, or other similar technologies known to those skilled in the art. This invention also encompasses the use of medical devices that are already equipped for wireless transmission using a protocol designed by the manufacturer, which is different from SPP. The novelty of the invention lies in the manner in which multiple medical devices, each of them having been made capable of wireless data communication, are networked at the bedside, and the manner in which the data communication sequence is initiated using the network resources and mobile computing device for performing various functions. These functions include the following;
Unambiguous identification of the person receiving the medical treatment;
Initiating and controlling the medical treatment by provisioning various services to the patient by a specific medical worker, such as on a secure data network with minimum opportunity for any unwanted, unintentional or undesired incursions by hospital staff or third parties;
Monitoring certain medical parameters of the patient during the treatment or longer times;
Gathering and delivering special messages from the medical or other devices such as alarms to the targeted recipients on assigned priority; and
Providing these services using equipment enabled for wireless data communication.

The description of an exemplary embodiment of the invention is presented here in the context of an installation around the bed of a patient receiving treatment in a medical facility which has installed wireless data network and a wired data networking infrastructure to support the wireless network, including the hardware and software components. This embodiment includes a bedside equipped with multiple medical devices which are capable of measuring the various physiological parameters of the patients, such as electrocardiogram signal, pulse oxymeter signal, blood pressure, temperature, respiration rate, and others as are known to those proficient in medical sciences. These devices are termed as sensors, or sensor units (SU), as they sense certain specific physiological parameter of the patient and convert the information in digital signal and allow the data to be exchanged with an external computing device following a certain protocol.

Another set of medical devices, termed actuators, are also placed around the bed of the patient. These actuators are devices which require an external input to initiate specific actions upon the person receiving the treatment. Examples of actuators include intravenous infusion pumps, or commonly known as 1V pumps, external oxygen flow control instrumentations, respirators, feed pumps, and other similar medical instruments. The actuator devices are designed to initiate certain action after receiving external commands, such as operate an IV pump to inject a specific medication in the body of the person at a preprogrammed rate, open the valve of an oxygen cylinder to allow the flow of controlled amount of the gas to a delivery system connected to a person, or control the rate of delivery of certain nutrients fed through a tubular mechanism from an entro-feeding pump. In addition, another set of external sensors or control devices may be deployed to monitor or control the ambient in the vicinity of the patient receiving treatment which is also covered by this invention. These devices are referred to as the environmental monitoring devices in this document. It also includes electronic devices capable of data communication which are gathering data from patient's surroundings, including those attached to the bed occupied by the patient to gather specific information about the patients and their activities. All these devices are included in the description of this invention.

### BRIEF DESCRIPTION OF THE DRAWINGS

FIG. 1 is a schematic representation of the connection between the medical device and the wireless transceiver.

FIG. 2 is a block diagram of the wireless transceiver module.

FIG. 3 shows a component arrangement of medical and communication equipment for provisioning at the bedside.

FIG. 4 is a flow chart of monitoring sequence using a local communication controller.

FIG. 5 shows a network configuration during set up.

FIG. 6 shows a network configuration during operation.

FIG. 7 shows a protocol describing the network setup procedure.

FIG. 8 shows a communication protocol during transit.

### DETAILED DESCRIPTION OF THE PREFERRED EMBODIMENT

The description that follows is presented to enable one skilled in the art to make and use the present invention, and is provided in the context of a particular application and its requirements. Various modifications to the disclosed embodiments will be apparent to those skilled in the art, and the general principals discussed below may be applied to other embodiments and applications without departing from the scope and spirit of the invention. Therefore, the invention is not intended to be limited to the embodiments disclosed, but the invention is to be given the largest possible scope which is consistent with the principals and features described herein.

It will be understood that in the event parts of different embodiments have similar functions or uses, they may have been given similar or identical reference numerals and descriptions. It will be understood that such duplication of reference numerals is intended solely for efficiency and ease of understanding the present invention, and are not to be construed as limiting in any way, or as implying that the various embodiments themselves are identical.

The invention teaches a method of performing the following functions:
Enable the medical personnel to positively identify the patient and obtain information regarding the recommended medical treatment for the patient;
Initiate a medical monitoring process using a single or a multiplicity of monitor on a secure data network with minimum opportunity for any unwanted, unintentional or undesired incursions by hospital staff or third parties;
Initiate a medical treatment using a single or multiplicity of actuators;
Remotely obtain information from the monitor(s) or the status of the actuator(s);
Receive input from the various monitor(s) and use it to control the actuator(s) in a manual or automatic fashion;
Record the details of the treatment imparted to the patient from the patient bedside;
Receive any alerts from the monitor(s) or the actuator(s) on a high priority basis over the network; and
Remote monitoring requires the use of a wireless data communication network which enables the medical person to receive the pertinent information remotely while walking in a hall or while dining inside the hospital or in any other area in the hospital which is covered by the wireless network.

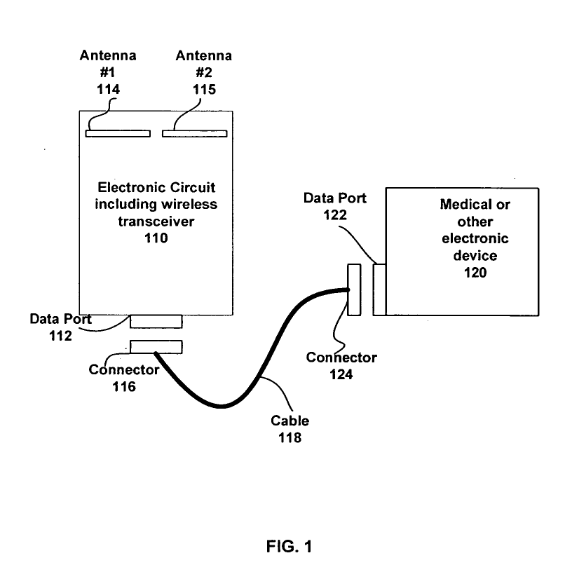

FIG. 1 shows the schematic diagram 100 of an electronic apparatus 110 which is designed to be connected to various medical and other devices 120 using a hard wired interface to exchange data with the medical device and transmit and receive them from the wireless transceiver contained in the device. This electronic circuit is referred to as wireless transceiver module, or WTU. It consists of a data port 112 at one end and a wireless antenna 114 at the other end for exchanging data from two sources simultaneously. In alternate embodiment of the invention, multiple antenna or transceivers are present on WTU. This diagram shows another transceiver or antenna marked as 115. (For wireless communication using infrared protocol such as IrDA or others, a diode will be used in place of antenna, as is known to those skilled in the art.) The data port 112 matches the recommended configuration of any one of the standard protocols used for data exchange. The medical and other devices 120 are normally designed to interface with a wired data network using a variety of different protocols. Most common of these protocols tends to be the serial data communication protocol commonly referred to as RS232. However, other protocols such RS422 may also be used. Additionally, protocols such as “USB” and “Firewire” may also be used. In certain instances, other data communication protocols such as Ethernet may also be deployed to exchange data with the local area or wide area data network. In other instances, data communication from the device may take place over parallel data bus. This invention covers all these protocols, and others offering the data communication capability from the medical device to the external data networks. On the other end of the WTU, a wireless data receptor or transmitter is located, which is an antenna in case of radio frequency wireless transmission, or diodes in case of infra-red wireless transmission. It will be understood by the foregoing that WTU describes any of the various wireless transmitter unit devices identified as 200, 319-328, 521-525, etc. described herein. It will also be understood by the foregoing that the terms medical devices or equipment refers to any of the various medical devices identified as 120, 310-318, etc. described herein.

A cable 118 is connected to the WTU 110 at the data port 112 using a connector 116. The other end of the serial cable is terminated in connector 124. This connector connects the cable to the medical device 120 at the data communication terminal on the medical device shown as block 122. Combination of WTU 110 and medical device 120 gives the medical device the ability to communicate wirelessly.

Additional functions are built in this WTU 110 to enable this electronic apparatus to make certain decisions locally based on the incoming data and the built-in rules, and also to reformat the information received from the medical device in the preferred format as taught by this invention before transmitting them over the network. The electronic circuit in WTU 110 has to be programmed prior to every use so that the communication parameters match those of the medical instruments. For communication using RS232 protocol, the WTU 110 serial interface has to be programmed to match parameters such as baud rate, stop bits, parity, handshake. WTU 110 is designed such that it can be programmed in two ways:
WTU 110 serial interface can be programmed by connecting it to an external programming device and downloading and storing the parameters in a non-volatile memory in the WTU 110, as is known to those skilled in the art; and
This electronic circuit can also be programmed by receiving instructions over the wireless link, and extracting the parameters from the wireless transmission and storing them in the non-volatile memory. An external wireless transceiver establishes communication with WTU 110 and downloads the programming data to the electronic circuit, and the required instructions to extract the data and send it to the program storage area for controlling the interface with the medical device.

This enables one single piece of hardware to communicate with a wide range of medical and other devices by simply reprogramming the serial interface and storing the parameters in the non-volatile memory of the WTU 110. Additionally, this electronic circuit is designed with intelligence to enable certain decision making functions to be embedded in the electronic circuit. It also is designed with the ability to store information.

Further references to medical devices in this disclosure imply the presence of this WTU 110 module to give them wireless data communication ability.

The WTU 110 is designed to comply with one or more wireless data communication protocols, such as 802.11, Bluetooth, Zigbee, IrDA, or other proprietary standards operating in other unlicensed or controlled frequency bands. WTU unit 110 also contains an additional identifier such as a paper sticker containing a serial number, a sticker containing a bar-code, or a RFID tag (object 140). In addition, the WTU 110 contains audio-visual indicators to provide physical confirmation of certain events (object 142).

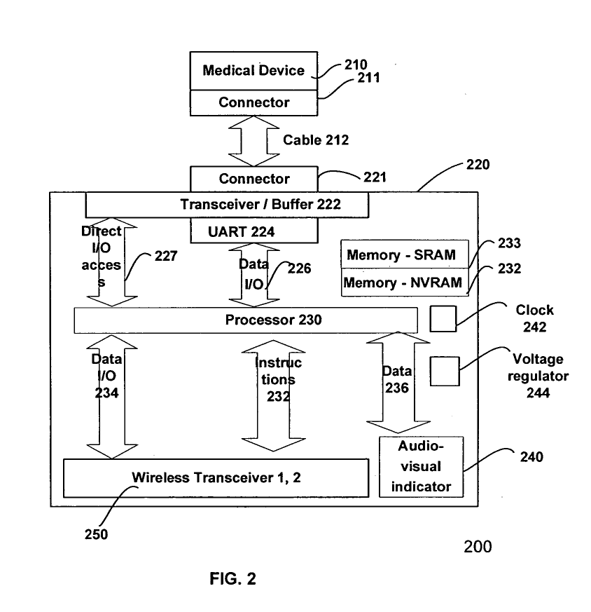

FIG. 2 shows the design of the wireless transceiver module, or WTU 110 in greater detail in 200. This module consists of various functional blocks contained inside the block 220. The WTU 110 contains a hardwired data interface which is used to connect to the medical device 120 or 210 external to the module. The data from the medical device is terminated on connector block 211. A cable 212 is used to connect the connector 211 to the corresponding connector 221 on the WTU 110. The physical specifications for the connectors may vary but the communication protocol of the medical device 210 and WTU 220 need to match for the two devices to communicate.

The connector 221 located on WTU 220 is terminated in a transceiver 222 which converts the electrical signals conforming to the data protocol to the internal operating voltage of the WTU 220. The output of the transceiver 222 is connected to an UART 224 for efficient operation of the WTU 220. The UART 224, or Universal Asynchronous Receiver-Transmitter, can be a controller chip or other circuit that processes data coming in and going between the transceiver buffer 222 and the central processor 230. The UART 224 feeds the data to a databus 226 which connects to a processor 230. In certain instances, the UART 224 and the data I/O bus 226 is included inside the same chip as the processor. Certain embodiment of this invention includes a direct connect 227 of certain I/O pins on the processor directly to the connector 227. The processor is also supported by memory modules, which consist of static random access memory 232 and non-volatile memory 233.

The processor 230 exchanges two types of information with the wireless transceiver 250. First, the processor controls certain programming functions required by the wireless transceiver. The programming instructions are exchanged over the bus 232. The process also exchanges data with the wireless transceiver 250 which is shown as bus 234. There is also an audio—visual indicator 240 supported by the processor, with a data bus 236 feeding to the A/V indicator 240.

Clock 242 and voltage regulator 240 set up timing and voltages for not only the processor 230 but also other devices associated with the WTU electronics 220.

The WTU 200 or 110 is designed so that it can support multiple radios operating on different protocols. These radios include radio frequency transceivers or infra-red transceivers, represented by the block 250.

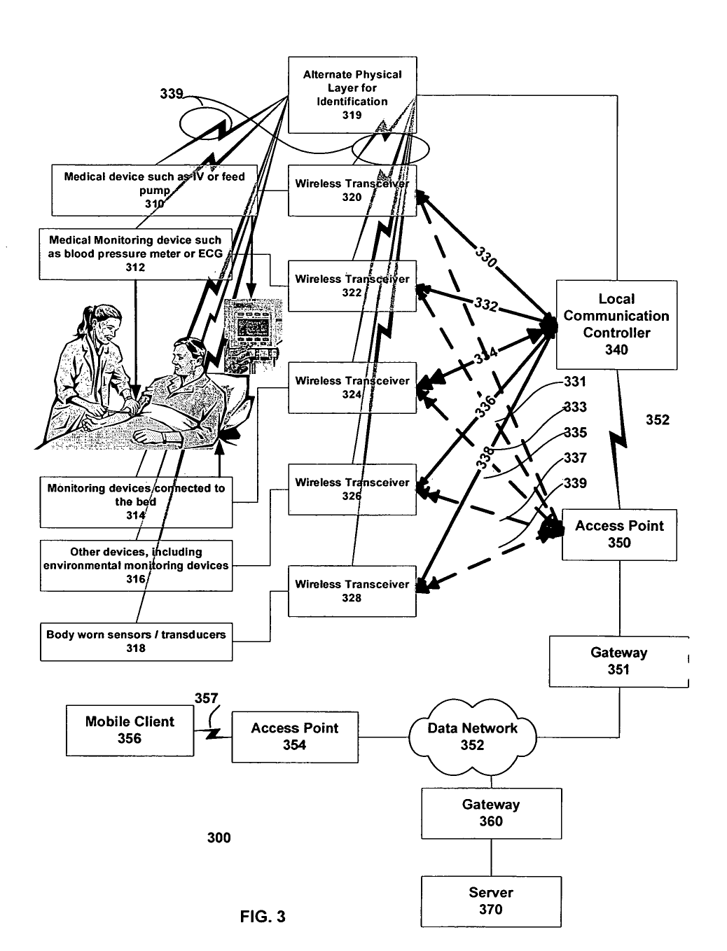

FIG. 3 shows the schematic arrangement 300 of various components of this invention for monitoring and receiving alarms from medical devices, including monitoring devices and actuators such as IV pumps, feed pumps and other medical instruments. The significant feature of this invention is the identification of the patient, care provider, medication, and medical equipment, and the operating parameters of the medical equipment in real time in order to insure that there is no error in delivering the prescribed treatment to the person. Medical devices include monitoring devices such as blood pressure monitors, pulse oxymeters, thermometers and actuator devices such as IV pump and the feed pump. These devices are represented as boxes 310 and 312. In addition, devices connected to the bed of the patient are shown in box 314. Example of these devices includes weighing machines, position sensors, etc. Environmental monitors such as humidity or temperature monitors are represented by box 316 and are also included in the embodiment of the invention. In addition, certain body-worn sensors and transducers are represented by box 318, such as electrocardiogram probes. All these devices are connected to independent and unique wireless transceiver modules (WTUs) marked as box 320, 322, 324, 326 and 328. The wireless transceiver modules are hardwired to the data port of the devices as described in FIG. 1. The wireless modules 320, 322, 324, 326 and 328 are capable of bidirectional communication using multiple protocols (Bluetooth, WiFi, Zigbee, Ultra-wide-band) and multiple physical medium (radio frequency, infra red) with a local communication controller (LCC) 340. These communication protocols are represented by objects links or arrows 330-338 respectively. The LCC 340 contains corresponding hardware and software to communicate using all these protocols. In alternate embodiments of the invention, multiplicity of WTUs 320, 322, 324, 326 and 328 use the same protocol to communicate with the LCC 340. It will be understood by the foregoing that LCC 340 describes any of the various local communication controller devices identified as 340, 510, etc. described herein.

A device 319 to physically identify the various devices 310-318 using alternate physical layer such as RFID or bar code is also included in the data network 300 around the patient bedside. This device is known as the reader which reads the tags placed on the various medical devices 310-318 and sends the information to LCC (340), who transmits it to the server 370 for identification and validation of the devices 310-318. The identification agent uses a protocol represented by object 339 to identify the medical devices 310-318 or the corresponding WTUs 320-328 using the tags 319 attached to those devices 310-318.

Local communication controller or LCC, represented by box 340, is designed to support any of multiple protocols represented by objects 330-338. The LCC 340 communicates with access point 350 using a protocol 352. In a certain embodiment of the invention, the protocol 350 is identical to any one of the protocols represented by object 330, 332, 334, 336, or 338, or any other protocol used for wireless communication. Alternate embodiment of the invention also includes this protocol to be a different protocol all together. The access point 350 is also designed to communicate with the WTUs (320-328) using protocols given by objects 331-339. The backbone of the access is connected to the local area or wide area data network 352 in the facility. An exemplary embodiment of the invention includes the access point 350 design to include a transceiver to support protocols 331-339, with each transceiver being accompanied by a bridge to Ethernet to connect to the data network.

An alternate embodiment of the invention is implemented with an access point 350 is connected to the local area or other data network 352 through a gateway 351, a network component known to those skilled in the art. A server (box 370) is connected to this data network. Another access point 354 shown in FIG. 3 is one of the multiple access points connected to the network 352. This exemplary embodiment shows this access point communicating with a mobile client device 356 using protocol 357. Alternate embodiment of the invention includes the protocol 357 to be identical to any one of the protocols represented by objects 330-338 or 352. In an exemplary embodiment of the invention, the protocol represented by objects 330-338, and 357 are Bluetooth, Zigbee, 802.11b/802.11g/802.11a, Ultra-Wide-Band, or any other suitable wireless data communication protocol. The details of these protocols are well known to those skilled in the art. It will be understood by the foregoing that the term access point describes any of the various access points identified as 350, 354, 540, 640, etc. described herein.

LCC 340 establishes communication with WTUs (320-328) to perform various tasks associated with the initiation of patient care such as identification of the patient, medicine, medical care provider, and the treatment, and storing the information generated at the bedside to the server. The details of data flow between LCC 340, the server 370, and various other nodes of the network 352 is described next.

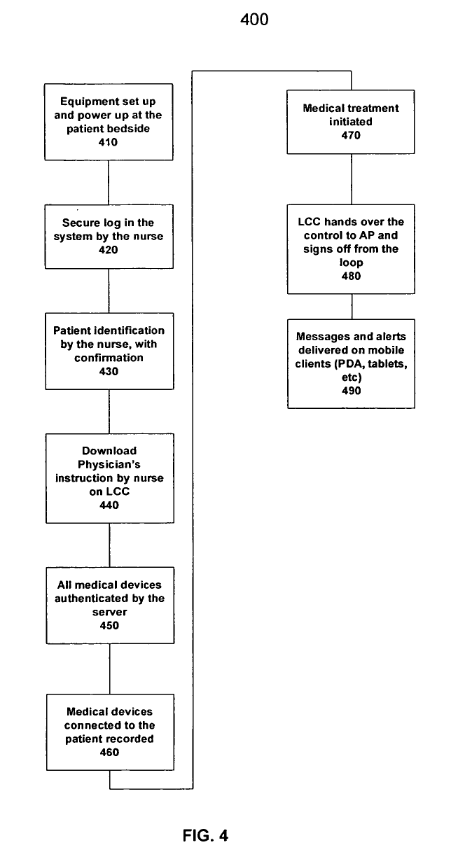

FIG. 4 shows the process flow 400 followed by the medical staff to perform a medical treatment of the present invention. In a preferred embodiment of the present invention, the invention is practiced following the “five rights, or the right patient, right drug, right dose, right route, and right time” known to those skilled in the healthcare sciences:

1. Clinical staff sets up the medical instruments at the bedside of the patient as the first step 410 in delivering the recommended treatment to the patient and arranges them at the bedside in the proper order. Each of these medical instruments is equipped with a WTU, which makes it capable of transmitting certain data wirelessly. Upon powering up, the radios in WTUs connected to various instruments enter the pre-programmed state. This exemplary embodiment includes radios conforming to the IEEE 802.11 standard. The WTUs are programmed to connect to the server in compliance with the security protocols of the establishment. All the devices are registered with the server.

2. The care provider is carrying LCC at the point-of-care with them. This LCC is wirelessly linked to the access point. Prior to providing any medical treatment, the care providers such as nurses identify themselves to system by connecting with the server using a browser in the LCC, and signing in the system using a password or an appropriate biometric sign-on procedure 420.

3. Identification of the patient is the first task to be performed by the care provider prior to initiating any medical treatment. Verification of the patient to insure that correct treatment will be imparted 430 is the next step in patient care. After completion of the previous step 420, patient identification step 430 is initiated by the server before initiating any treatment. Server downloads various instructions as a web page on LCC to guide the care provider. Identification of the patient involves obtaining information about the patient and sending them to the server so that the patient identity can be positively confirmed with the entries in the server database. Exemplary procedures to identify a patient include downloading a list of the patients cared for by the specific staff member, and checking the appropriate entry on the screen of the LCC to identify patients. The information presented on LCC includes name of the patient, physical location of the patient, a picture of the patient, and any other identifiers provided by the authorities. Alternate methods for identifying a patient includes reading a patient worn identification device such as a barcode tag or a RFID tag using an appropriate reader. (The patient worn identification device is already indexed to the patient in the server database at the time of admitting the patient in the hospital.) This data is gathered by LCC and is transmitted to the central server over the wireless network from the bedside of the patient prior to initiating any medical process. An alternate embodiment of this step 430 in the invention also includes gathering of picture of the patient using a camera connected to LCC, and sending the picture to the server to receive confirmation by performing facial recognition. Additional biometric measures such as finger print or speech recognition are also covered by alternate embodiments of the invention to confirm the patient identity.

4. The details of the physician's instructions and the required medical devices to fulfill them are downloaded in the LCC at the bedside of the patient after the patient has been positively identified by the system. An embodiment of this invention includes the process of identifying the instruments being used for carrying out the medical treatment. The configuration of the medical instruments dedicated to a certain bedside to provide medical treatment is named “bedside area network” or BAN. The list of the instruments in a certain BAN is generated with input from the bedside and stored in a table in the server. The medical staff is able to extract instructions from the physician or the appropriate authorities to initiate certain medical treatment, monitoring schedule, and the corresponding list of instruments from the server. Alternately, the instructions can also be written on paper or orally given by the physician in which case the selection of the instruments is made by the medical staff manually.

5. An LCC is carried to the bedside by medical personnel. It is already connected with the server and has a list of the medical device types required for the treatment. LCC performs the first level of validation of the medical instruments at the bedside, as shown in step 450. Any violations are displayed on the screen and message is displayed suggesting that certain device types are not required for treatment, or certain devices are missing. Further details of this process are presented further in this documentation.

6. Complying with the instructions, the medical instruments are arranged at the patient bedside and connected to the patient as shown in step 460. Exemplary arrangements include connecting a blood pressure cuff around the arm of a patient, placing an infra-red sensor on the finger of a patient for monitoring blood oxygen, attaching probes for gathering electrocardiogram traces, injecting a syringe in a patient in preparation to deliver a medicine through a pump, and others known to those skilled in the medical sciences. A combination of these procedures is also covered by alternate embodiments of the invention. As these devices are powered up, their physical arrangement is recorded and verified by the system. Details of the verification procedure are described further in this document.

Based on the recommended treatment, the medical personnel connect various medical instruments with the chemical agent including medicine, gases, or other ingredients to the patients in the recommended manner, as shown in step 460. In an alternate embodiment, the connection between the medical devices and between medical devices and the patient is entered as a schematic diagram on the LCC. It is recorded by the server and maintained as a part of the patient medical record. This block diagram contains the configuration of medical devices with respect to the patient. An exemplary embodiment of the invention includes a schematic diagram of most commonly used configurations such as use of a single, double, or triple drip IV, combination of IV with oxygen saturation monitoring available to the medical staff on LCC in a drop-down menu. At the same time, a software package is provided on LCC where more complex arrangement of medical equipment connected to the patient can be recorded saved as a part of the patient's medical record.

In the preferred embodiment of the invention, the exchange of information between LCC and the medical devices takes place over a uniform wireless protocol, determined by the WTU connected to them. An alternate embodiment of the invention covers the instance where the various WTU are communicating with the medical devices using different communication protocols. In order to manage the communication with the different medical devices using different protocols, an application level protocol is used by this invention which defines a uniform method for exchanging data between the WTU and the LCC. Hence, the datagram gathered by a WTU from a blood pressure meter from manufacturer A or B is consistent. Also, the data delivered from a WTU connected to IV pump using a wireless link based on 802.11b or Bluetooth have exactly the same structure upon reaching LCC. This data packet meets the specification of Sensitron Personalization Protocol (SSP) as described in detail in U.S. patent application Ser. No. 10/850,527 completely assigned to the Applicant herein having filing date May 19, 2004.

7. After the preparations have been completed, the medical process such as drug injection through the pump is initiated manually by the care provider and has the time recorded by LCC. Alternately, the instructions to initiate the drug injection are sent as a command from LCC to the two WTU connected to the corresponding IV pumps 470.

8. After initiating the monitoring process, the WTUs from the medical devices communicate with the wireless data network without any need for supervision. In such instances, the role of LCC is obviated or eliminated or otherwise minimized after the monitoring process is started. LCC signs off from the BAN. The WTUs connected to the pumps and other medical devices communicate directly to the server, as shown in step 480 through the access point of the local area or other data network.

9. Finally, the information from the server is delivered to any client device connected to the wired or wireless network. These client devices range from a desktop notebook, a wireless notebook or a PDA, a pager capable of receiving text or numerical messages, or a cellular phone capable of receiving textual messages as represented in step 490. Any error generated during transmission is handled by the server and the WTU using different protocols. Upon a break in the network availability, the server informs the medical staff using email, audio-visual indicators or any other suitable means such as a synthesized spoken message that the network connection to a specific patient monitors has become unavailable, and certain corrective actions need to be taken to remedy the problem. The WTU, on the other end, is programmed to record data during network outage and store the data locally while the network connection is unavailable. WTU is also programmed to present an audiovisual display to represent a lost network connection. Any error messages generated by the medical devices are also stored by the WTU and given priority over data storage. They are also delivered with priority to the server when the connection is re-established as shown in step 490.

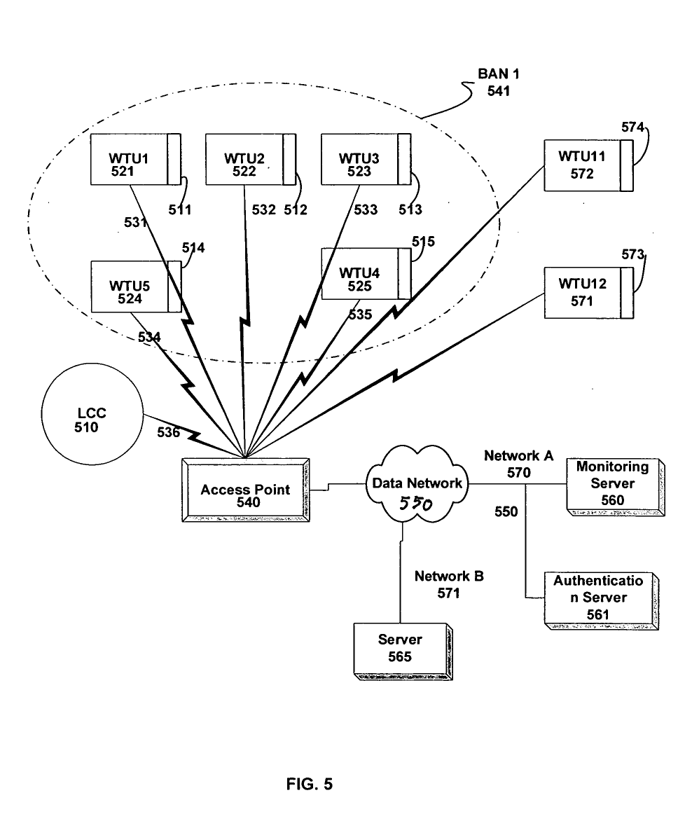

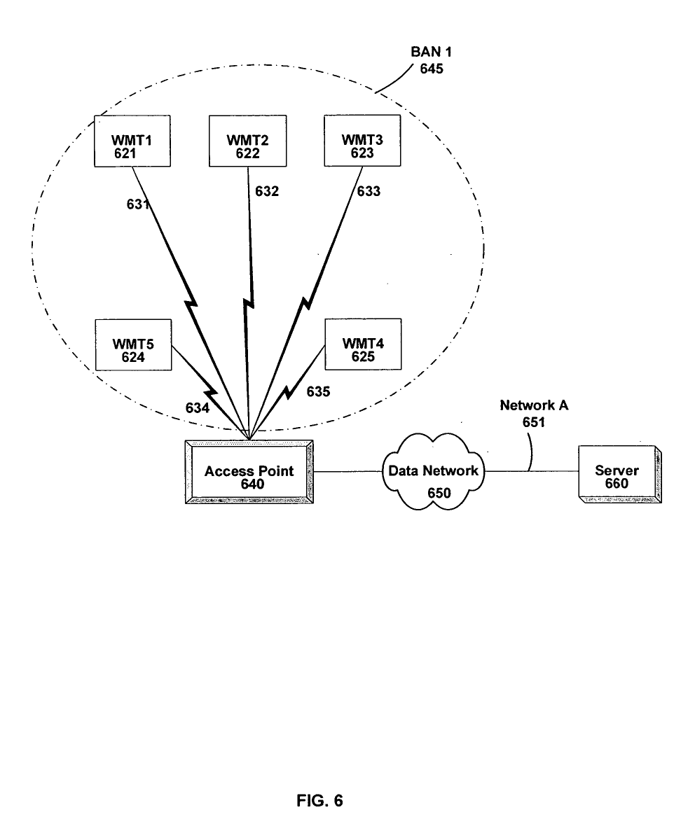

The process of establishing the network of wirelessly enabled medical devices at the bedside or the BAN, including verification with the instructions and hospital rules is described here with the help of FIGS. 5 and 6.

FIG. 5 shows the network diagram of the wirelessly enabled medical devices at the bedside at the onset of medical treatment. The medical devices and their corresponding WTUs are represented by blocks 521-525. Each WTU has an associated AMI represented by objects 511-515, respectively. In the present application, AMI is “Alternative Media Identifier”, i.e., the communication using a different media or radio standard to identify a certain part. The method is used to identify a certain device, instrument or piece of equipment using AMI implemented in a device such as a bar code, RFID tag, or simply reading its serial number previously identified. Each of these medical devices and their associated WTUs communicates with the access point 540 using wireless protocols 531-535, respectively. These devices form a BAN 541. In a preferred embodiment, two WTUs belonging to other BANs are shown in FIG. 5, represented by objects 571 and 572. The area associated with AMIs given by objects 573 and 574 respectively, communicating to the access point 540 also. This invention covers all the combination of wireless protocols represented by objects 531 to 535. This includes protocols such as 802.11b, 802.11 g, 802.11a, Bluetooth, Zigbee, UltraWideBand, and other proprietary protocols established by various manufacturers.

Prior to commencement of any medical treatment process, each of the WTUs are preprogrammed in a controlled environment to perform the following tasks:

1. Data communication interface of the WTUs is programmed to contain the appropriate parameters to communicate without any error to the corresponding medical devices.

2. Each WTU is programmed to communicate directly with the monitoring and data server (object 560). This is required to insure security of the wireless network. The network ID (SSID) and name of the server are stored in the non-volatile memory portion such as 232 in FIG. 2 of the WTU prior to the installation. It will be understood that SSID stands for Service Set Identifier. In various protocols, SSID specifies which 802.11 network you are joining. Some systems allow you to specify any SSID as an option so you can join any network. The SSID is a token which identifies an 802.11 (Wi-Fi) network. The SSID differentiates one WLAN from another, so all access points and all devices attempting to connect to a specific WLAN must use the same SSID.

3. An encryption key assigned to the WTU is stored in the WTU. A corresponding key for decrypting data is also stored in a table generated in the server. The MAC (media access control) address of the WTU, or the address of the WTU device on the network, is also retrieved by the server and stored in the table as an attribute for the WTU. Alternately, a certificate of authentication is stored in the WTU.

4. During programming, the AMI of the WTU is recorded by an appropriate means and stored in the server along with the encryption key assigned by the server to the WTU.

This data is downloaded in the WTU through the wireless (802.11) based interface in a protected, shielded environment. Alternately, it is downloaded in the WTU through the wired data port 112 as shown in FIG. 1.

The authentication, monitoring and data storage is carried out by the server 560 connected to network 570 over the local area or wide area data network 550. Alternately, an authentication server 561 is connected to network 570 to perform the authentication of each WTU joining the network. Another network 571 is also shown here which connects server 565 to the LAN.

Each WTU and LCC is programmed to communicate with the same server (560) and the same network (570). This network connection is set up when the LCC, in the form of a mobile computing device such as a laptop computer, notepad, tablet or PDA/communication device, is brought in the vicinity of the patient bedside. LCC is programmed to access the server using a browser-type operating program/interface. Thus, no additional software is needed on LCC. This feature makes it possible to use a wide variety of computing hardware for LCC, since they only need to host a web browser locally.

FIG. 6 shows the network at the completion of BAN with the monitoring process in full swing. It only shows the WTUs (objects 621-625) that are a part of BAN 645. These WTUs are communicating with the access point 640 using protocol 631-635. The access point 640 is connected to the LAN 650. A preferred embodiment of the invention comprises a server 660 connected through the network 651. It should be noted that the LCC is not of a part of this network during the regular monitoring process.

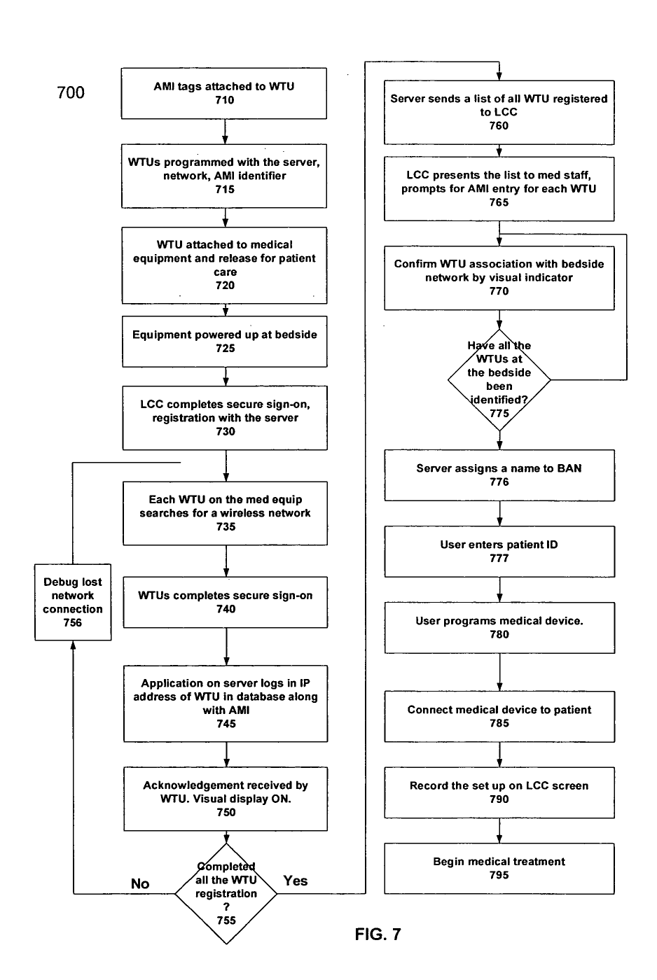

The protocol 700 used for establishing a BAN is described next in FIG. 7. First step in establishing a BAN successfully is to attach an AMI to each WTU. The details of AMI tags have been described herein. This physical process is shown in step 710.

The next step in defining the BAN is to associate a WTU with a medical instrument and program the WTU to connect to a certain network and a server at the establishment upon power up, as shown in step 715. A list of all the information to be stored in the WTU and the server is listed as a part of description of FIG. 5 earlier. The programming of WTU takes place using the built-in radio in the WTU in a shielded environment to prevent unauthorized access during programming. Alternately, it takes place using the hardwired data interface on the WTU. Programming is carried out sequentially or simultaneously on all WTUs. An application is run on a server which provides necessary instructions to the personnel programming the WTUs. The interface programming instructions are derived from a library built-in the server cross-referenced with the type of medical device. Upon recognition of the medical device type, the interface programming instructions are downloaded in WTU which become active upon next power up sequence.

As described above, the parameters downloaded in the WTU are the names and addresses of the network and the server where the WTU is required to connect upon powering up. A secret key to authenticate itself is also downloaded in the WTU. A table of the MAC address of the WTU, its AMI, and its secret key SSID is stored in the server. These identifiers, or AMI, include a serial number of a WTU which is read by the medical staff and entered manually, entered as a picture taken from a miniature camera, read using a bar-code reader, or using a RFID tag reader. Alternately, the MAC address of the WTU is encrypted and transformed in a barcode or another identifier which is also attached to the outside of WTU.

Next, the WTU is physically attached to the medical device as shown in step 720. A preferred embodiment of the invention includes the identity of the medical device to be stored in the table along with the identity of the WTU that it is associated with. This information regarding the medical device is stored in the table along with the identifiers for WTU. This wireless enabled WTU is released for patient care.

The wirelessly enabled medical equipments are brought to the bedside in preparation for providing medical treatment and powered up, as shown in step 725.

An LCC is brought to the bedside, operated by a medical personnel. A secure sign on is completed by the medical person using the LCC, as shown in step 730.

Simultaneously, each WTU attached to the medical equipment starts searching for a network upon power up, as shown in step 735.

Each WTU checks the SSID of the network and matches with the SSID of the network name stored in the WTU. Upon verification of the SSID address, WTU sends a request to join the network. The access point and the authentication server execute the protocols for verifying the WTU identity using the wireless security protocols such as WEP, 801.1x, EAP, LEAP, PEAL, EAP-TLS, and others. The WTUs securely sign on to the network using the key, the name of the network and the server. This is shown in step 740.

Upon completion of the authentication process, the WTU requests connection with the server next. An application running on the server logs in the mac address of the WTU, its secret key, and its IP address. This application adds the WTU data to a table and enters its AMI in the table. It is shown in step 745.

As each client (a WTU) is given an IP address, a confirmation message is sent to the it by the server. Upon receipt of the confirmation message from the server, a visual display is switched on the WTU to confirm that it has been registered. It is shown in step 750.

A visual inspection is made by the medical person setting up the network to confirm that all the WTUs connected to each medical device have been registered by the network (step 755). In the event that any one of the WTUs attached to a medical device is not turned on, a debug process is initiated (step 756). In an exemplary embodiment, an application is launched on LCC which queries the network nodes, including the access point, and determines the reason for failure of the registration process. The details of this network debug procedures are well known to those skilled in the art of wireless network management and are beyond the scope of this disclosure.

In the event that all the WTUs confirm registration at the server, a list of all the active WTUs is presented to the LCC (step 760). This list is a superset of the WTUs attached to the medical devices required for treatment at the bedside.

The application running on the server extracts the list of various WTUs and delivers the list to LCC where it displayed in a table (step 765).

In order to identify only the WTUs associated with the specific patient, the medical staff is prompted to enter the AMI of each WTU (step 770). This AMI is entered by typing in the serial number, capturing and transmitting the visual image of the serial number tag, reading the bar-code on the WTU, reading RFID tag on the WTU, or any other appropriate method of identification of WTU. Entry of the AMI is recorded by LCC and transmitted to the server. Verification application on the server checks each entry of AMI against the entries present in the table which have been made during registration of WTU. At the end of identification process, all the WTUs are scanned by the medical staff and the entries are stored and verified in the server. Correspondingly, the list on the screen of the LCC is updated in a textual or graphical format which contains all the WTUs associated with the patient. Upon positive verification by the care provider, the association between the medical devices and their corresponding WTUs and the patient is finalized. A visual indicator is turned on the WTM to signify that it has been associated with a bedside area network.

This process is carried out sequentially for all the WTUs at the bedside (step 775).

Upon finalizing the network nodes at the bedside being used for the patient, the server assigns a unique identifier to BAN (step 776).

LCC requests a patient identifier and associates it with the BAN (step 777) by entering the relevant information. Patient identifier include, but are not limited to, such indices as patient name or other identifiers made available by the institution, patient ID using a manual or automated entry of an index on the wrist band including bad-code tag or a RFID tag, or a biometric identifier for the patient. Other information such as patient location, identification of medical staff is also entered in the system. In an alternate embodiment of the invention, the location is captured by a location management system installed as a part of the wireless network or as a separate service, and the identification of the medical staff is also entered by reading bar-code or an RFID tag on their badges.

In an alternate embodiment of the invention, the patient ID or the location of the patient is downloaded to the WTU during the initial programming of the WTU. Upon reaching the patient bedside, they register with the server as shown in step 755. For additional confirmation of presence of the WTU proximate to or in the essentially immediate vicinity of a patient, the patient ID is matched with the corresponding ID stored in the server. This allows only the WTUs preprogrammed for use at a certain patient's bedside to be recognized by the server as part of the BAN.

LCC accesses the recommended treatment instructions by the physicians or others in charge of medical care from the server for the given patient from the server (step 780). For the medical monitoring devices, the recommended upper and lower limits of vital signs are accessed from the instructions and entered in the LCC. Alternately, these limits are imported from another database on the network. These limits are stored in the patient database, and are used for generating alarms.

If required, the medicines connected to the IV pumps are entered in the records by an application on LCC. One embodiment of the invention teaches the process of recording the medication on the selected pump by entering the identity of the medication using keyboard manually. It can also be selected from a list of the medications which are recommended by the physician and which have been earlier downloaded in the LCC. This medication can also be identified by means such as RFID to the database.

After identification of the pump and the medicine, the rate of delivery of the medicine through the IV pump is programmed in the pump. Alternately, the rate of delivery of medicine is done manually by the medical staff. Physician's instruction for drug delivery to a patient through the IV pumps are input in the system by direct import from a database or entered manually by the medical staff at the point-of-care on LCC. This invention also includes the translation, if needed, from the recommended dose to actual numbers to be entered in IV pump. Exemplary embodiment includes the case where the drug dose is recommended in terms of weight of drug per unit weight of patient body weight. The weight of the patient is obtained from the bed, if the bed is provisioned with the sensors to perform the task, or obtain the data from patient database alternately. After final calculations, the number to be entered in the IV pump is presented to the medical staff. In yet another embodiment of the invention, this number is directly exported from the LCC to the IV pump. The pump in set automatically to deliver these dose numbers to the patient.

This process is repeated to identify the second IV pump, including identification of pump, verification of the medicine and the rate of delivery of medicine from the second pump.

The medical devices are connected to the patient next, as shown in step 785.

The next step is to map the connection of various medical instruments and the medical device to the patient on the screen of LCC, as shown in step 790. The connection of the medical devices (sensors, actuators) to the patient is described by a text based system, where each medical device (represented by its WTU) is highlighted and a drop down menu allows the medical staff to choose the selected method of connection. Exemplary embodiment includes a blood pressure machine with a drop down menu containing the option of left or right arm, sitting or lying down. Another example includes a pulse oxymeter with the options to connect the infra-red sensors to a specific finger of the patient. Any oxygen being given to the patient at that time is also available for recording through a manual entry, or directly read from oxygen flow meter. The medication delivered to the patient through the IV pump is also recorded by drop down menus which contains a list of possible locations on the human body where the syringe can be inserted.

After the system has been programmed and verified by the medical staff, the application on LCC clears the medical treatment to begin (step 795). The beginning of the medical treatment includes the start of delivery of medication, start of nutrient delivery, and recording of vital signs. At this point, the server stores the details of the network nodes, including their IP addresses in the patient database. For further treatment, server maintains bidirectional communication with the network nodes and receives data from them. Any loss of communication between the server and any network node raises an alarm which is escalated according to established policy of the medical institution. LCC is allowed to sign off and disconnect from the server.

Any further change in the setting of any of the medical devices by the medical staff is allowed only by logging on to the server, entering the patient identifier and the location, entering the change in the patient database, and making the corresponding change in the medical device setting after proper authorization. Any attempt to change the setting of any medical device without prior authorization is delivered to the server immediately and raises an alarm which is delivered to the medical personnel as per the hospital policy.

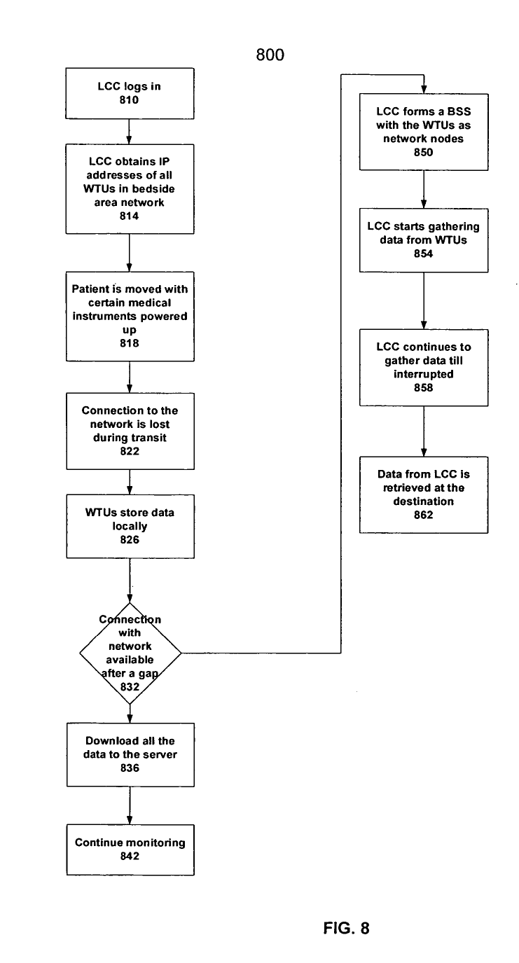

FIG. 8 shows a communication protocol during transit, i.e., the operation and functioning of the system during a physical move of a patient from one location to another. This is facilitated by a Transient Communication Controller TCC. This is a mobile computing device with capability to connect with all the WTUs associated with the various medical instruments. This logical unit TCC is used by the medical staff to log in the server from the patient bedside, and enter the relevant patient information to identify the patient and the medical devices belonging to the bedside area network being relocated (step 810).

The TCC downloads the IP addresses of the WTUs connected to the medical instrument which form the bedside network from the server and retains a list of the medical devices which are to be moved (step 814). TCC is empowered to modify the list of the equipment being moved with the patient. In an exemplary embodiment, three IV pumps are attached to the patient but during the move, only one IV pump releasing a certain medicine is carried with the patient. Such variations are recorded on TCC.

As the patient and a specific few medical devices are moved, they keep on transmitting the data to the server over the network although the access points will change from one location (step 818).

In the instance that the network becomes unavailable for a certain period of time (step 822), the WTUs are designed to store the data locally (step 826). This gap in the network availability ranges from one seconds to ten hours and it can be extended by increasing the storage capacity of the WTU by adding more memory.

The WTUs transmit all the data to the server when the network becomes available and the WTUs are able to connect to the server (step 836). After that, medical treatment is continued and the data transmission enabled (step 840).

In the event that the server is not available for a period beyond a certain limit as described above, or upon receiving a user prompt (step 850), TCC takes over the role of a server and forms a BSS (basic server set) with the bedside WTUs while the patient is being moved.

A preferred embodiment of the invention considers such a case when the patient is being moved from a specific medical facility to another by an ambulance. The TCC keeps storing all the data in the storage unit associated with it (step 854). During the move, TCC also displays the data on an application running on it.

The data storage by TCC continues till it is interrupted by an external input (step 858).

TCC connects to the network and allows the care provider to access the stored data after authentication (step 862).

Unless defined otherwise, all technical and scientific terms used herein have the same meaning as commonly understood by one of ordinary skill in the art to which the present invention belongs. Although any methods and materials similar or equivalent to those described can be used in the practice or testing of the present invention, preferred methods and materials are now described. All publications and patent documents referenced in the present invention are incorporated herein by reference.

While the principles of the invention have been made clear in illustrative embodiments, there will be immediately obvious to those skilled in the art many modifications of structure, arrangement, proportions, the elements, materials, and components used in the practice of the invention, and otherwise, which are particularly adapted to specific environments and operative requirements without departing from those principles. The appended claims are intended to cover and embrace any and all such modifications, with the limits only of the true purview, spirit and scope of the invention.

1. A wireless transceiver unit for wireless communicating instructions and data between a medical device or electrical monitoring equipment and a data network, the wireless transceiver unit comprising:
an electronic interface for communication of data collected from a medical device or other electrical monitoring equipment utilizing a predetermined operating control and data communication protocol;
a central processor which converts the data collected from the medical device or other electrical monitoring equipment into signals conforming to a predetermined wireless data communication protocol;
a wireless transceiver for receiving the signals conforming to the predetermined wireless data communication protocol and transmitting the signals to a secure, data network.

2. The wireless transceiver unit of claim 1 in which the electronic interface comprises a physical, electronic connector for connecting to the medical device or other electrical monitoring equipment utilizing the predetermined operating control and data communication protocol.

3. The wireless transceiver unit of claim 1 in which the predetermined operating control and data communication protocol associated with the medical device or other electrical monitoring equipment and electronic interface is a protocol selected from the group consisting of serial data communication protocol, RS232, RS422, Ethernet, USB, firewire, parallel data communication protocol, and any other manufacturer standard or proprietary protocol.

4. The wireless transceiver unit of claim 1 further comprising a transceiver buffer positioned intermediate the electronic interface and the central processor which converts the electrical signals conforming to the data protocol utilized by the medical device or other electrical monitoring equipment to the internal operating voltage of the WTU.

5. The wireless transceiver unit of claim 4 further comprising a Universal Asynchronous Receiver-Transmitter or similar circuit that processes data coming and going between the transceiver buffer and the central processor.

6. The wireless transceiver unit of claim 1 further comprising direct connect of I/O pins on the processor directly to the electrical interface.

7. The wireless transceiver unit of claim 4 further comprising direct connect between the processor and the transceiver buffer.

8. The wireless transceiver unit of claim 1 further comprising memory modules which support the processor.

9. The wireless transceiver unit of claim 8 in which the memory modules are selected from the group consisting of static random access memory modules and non-volatile memory modules.

10. The wireless transceiver unit of claim 1 in which the processor exchanges programming functions information with the wireless transceiver.

11. The wireless transceiver unit of claim 1 in which the processor exchanges data collected from the medical device or other electrical monitoring equipment with the wireless transceiver.

12. The wireless transceiver unit of claim 1 in which the predetermined wireless data communication protocol utilized by the wireless transceiver is a protocol selected from the group consisting of Bluetooth, Zigbee, 802.11b, 802.11g, 802.11a, Ultra-Wide-Band, or any other wireless data communication protocol.

13. The wireless transceiver unit of claim 1 further comprising a visual indicator supported by the processor.

14. The wireless transceiver unit of claim 1 in which the visual indicator is a light.

15. The wireless transceiver unit of claim 1 in which the wireless transceiver can support multiple radios operating on different wireless protocols.

16. The wireless transceiver unit of claim 1 in which the wireless transceiver is selected from the group consisting of radio frequency transceivers and infra-red transceivers.

17. A system for wireless communication of collected data between multiple medical and non-medical devices at the bedside of a patient and a data network, the system comprising:
one or more medical or non-medical data collection devices located within a bedside area network, wherein each of the one or more medical or non-medical data collection devices is equipped with a wireless transceiver apparatus for wireless electronic data communication using a wireless communication protocol; and
a data network which collects data and monitors each of the one or more medical or non-medical data collection devices by communicating with the wireless transceiver apparatus using a wireless communication protocol.

18. A system for monitoring processes and receiving alarms from medical devices and other monitoring equipment or data collecting sources, the system comprising:
one or more medical devices such as an IV liquid or nutrient feed or drug pump to be monitored and for providing data, each of the one or more medical devices equipped with an apparatus for electronic data communicating using standard protocols to allow the users to exchange information with these devices over hard wired connections, each of the one or more medical devices further connected to a wireless transceiver unit capable of bidirectional communication with a local communication controller using wireless communication protocol, each of the one or more medical devices further equipped with one or more alternate physical layer identification devices;
optionally, one or more patient monitoring devices such as a blood pressure monitor or ECG connected to the patient, each of the one or more patient monitoring devices equipped with an apparatus for electronic data communicating using standard protocols to allow the users to exchange information with these devices over hard wired connections, each of the one or more patient monitoring devices further connected to a wireless transceiver unit capable of bidirectional communication with a local communication controller using wireless communication protocol, each of the one or more patient monitoring devices further equipped with one or more alternate physical layer identification devices,
optionally, one or more bed monitoring devices such as a weight sensor or position sensors connected to the patient's bed, each of the one or more monitoring devices equipped with an apparatus for electronic data communicating using standard protocols to allow the users to exchange information with these devices over hard wired connections, each of the one or more bed monitoring devices further connected to a wireless transceiver unit capable of bidirectional communication with a local communication controller using wireless communication protocol, each of the one or more bed monitoring devices further equipped with one or more alternate physical layer identification devices,
optionally, one or more environmental monitoring devices such as an air temperature or relative humidity gauges or sound monitors located within the nearby environment of the patient, each of the one or more environmental monitoring devices equipped with an apparatus for electronic data communicating using standard protocols to allow the users to exchange information with these devices over hard wired connections, each of the one or more environmental monitoring devices further connected to a wireless transceiver unit capable of bidirectional communication with a local communication controller using wireless communication protocol, each of the one or more environmental monitoring devices further equipped with one or more alternate physical layer identification devices,
optionally, one or more body-worn sensing devices such as one or more electrocardiogram probes connected to the body of the patient, each of the one or more sensing devices equipped with an apparatus for electronic data communicating using standard protocols to allow the users to exchange information with these devices over hard wired connections, each of the one or more body-worn sensing devices further connected to a wireless transceiver unit capable of bidirectional communication with a local communication controller using wireless communication protocol, each of the one or more body-worn sensing devices further equipped with one or more alternate physical layer identification devices,
a local communication controller comprising the required hardware and software to communicate with each of the wireless transceiver units according to their respective wireless communication protocols;
a reader for each of the alternate physical layer identification devices, the reader capable of independently communicating the identification of the one or more medical or non-medical data collection devices to the local communication controller for purposes of identification and validation of the one or more medical or non-medical data collection devices; and
an access point for providing a secured gateway for communications directly between a wireless transceiver and a data network, the access point accessed by establishing primary communication with the local communication controller.

19. The system of claim 18, further comprising a mobile client, such as for receiving alarms or other data from the one or more medical devices, the mobile client having a wireless connection between the client and another access point to the data network.

20. A method of providing a medical treatment comprising the following steps:
Setting up the medical instruments at the bedside of the patient in a predetermined order, each of the medical instruments equipped with a WTU capable of transmitting certain data wirelessly to connect to the server of a data network in compliance with security protocols of the establishment;
Identifying the care provider to the system by connecting with the server using a browser in a local communication controller using a secure sign-on procedure;
Verifying the identity of the patient to insure that correct treatment will be imparted;
Identifying the details of the physician's instructions and the required medical devices to fulfill them at the bedside of the patient;
Authenticating all the medical devices in the bedside area network by the server with an LCC;
Connecting the medical instruments arranged at the patient bedside to the patient according to the physician's instructions;
Initiating the medical process either manually by the care provider or upon a command from the LCC to the one or more WTUs connected to the corresponding medical devices while recording the process data to the LCC;
Transferring communication and control of the medical devices completely from the LCC directly through an access point to the wireless data network; and
Delivering process or alarm data from the server to any client device connected to the wireless network, the client devices selected from the group consisting of a desktop notebook, a wireless notebook or a PDA, a pager capable of receiving text or numerical messages, or a cellular phone capable of receiving textual messages.

21. The method of claim 20, wherein the step of verifying the identity of the patient further comprises the following step:
Downloading from the server various instructions as a web page on the LCC to guide the care provider.

22. The method of claim 20, wherein the step of verifying the identity of the patient further comprises the following step:
Sending information to the server so that the patient identity can be positively confirmed with the entries in the server database.

23. The method of claim 20, wherein the step of verifying the identity of the patient further comprises the following step:
Downloading a list of the patients cared for by a specific staff member and checking the appropriate entry on the screen of the LCC to identify patients.

24. The method of claim 20, wherein the step of verifying the identity of the patient further comprises the following step:
Reviewing the name of the patient, physical location of the patient, a picture of the patient, and any other identifiers provided by the authorities.

25. The method of claim 20, wherein the step of verifying the identity of the patient further comprises the following step:
Reading a patient worn identification device such as a barcode tag or a RFID tag using an appropriate reader.

26. The method of claim 20, wherein the step of verifying the identity of the patient further comprises the following step:
Gathering the information via an LCC and transmitting the data to the central server over the wireless network from the bedside of the patient prior to initiating any medical process.

27. The method of claim 20, wherein the step of verifying the identity of the patient further comprises the following step:
Obtaining a picture of the patient using a camera connected to LCC, and sending the picture to the server to receive confirmation by performing facial recognition.

28. The method of claim 20, wherein the step of verifying the identity of the patient further comprises the following step:
Using biometric measures such as finger print or speech recognition to confirm the patient identity.

29. The method of claim 20, wherein the step of identifying the details of the physician's instructions and the required medical devices further comprises the following step:
Identifying the instruments being used for carrying out the medical treatment.

30. The method of claim 20, wherein the step of identifying the details of the physician's instructions and the required medical devices further comprises the following step:
Configuring the medical instruments dedicated to a certain bedside in a bedside area network.

31. The method of claim 20, wherein the step of identifying the details of the physician's instructions and the required medical devices further comprises the following step:
Generating the list of the instruments in a bedside area network with input from the bedside paired with data stored in the server.

32. The method of claim 20, wherein the step of identifying the details of the physician's instructions and the required medical devices further comprises the following step:
Allowing the medical staff to extract instructions from the physician or the appropriate authorities to initiate certain medical treatment, monitoring schedule, and the corresponding list of instruments from the server.

33. The method of claim 20, wherein the step of identifying the details of the physician's instructions and the required medical devices further comprises the following step:
Providing the instructions written on paper or orally given by the physician and manually selecting the instruments.

34. The method of claim 20, wherein the step of authenticating all the medical devices in the bedside area network further comprises the following step:
Using a LCC carried to the bedside by medical personnel already connected with the server and containing a list of the medical device types required for the treatment wherein the LCC performs the first level of validation of the medical instruments at the bedside.

35. The method of claim 20, wherein the step of authenticating all the medical devices in the bedside area network further comprises the following step:
Displaying any violations or messages on the screen suggesting that certain device types are not operating properly, not required for treatment, or certain devices are missing.

36. The method of claim 20, wherein the step of connecting the medical devices to the patient further comprises the following step:
Connecting a monitoring device to a patient, the monitoring device selected from the group consisting of vital signs monitoring devices, blood pressure monitoring devices, infra-red or other blood oxygen sensors, blood sugar monitoring devices and probes for gathering electrocardiogram, encephalogram or other electronic traces.

37. The method of claim 20, wherein the step of connecting the medical devices to the patient further comprises the following step:
Injecting a syringe in a patient in preparation to deliver a medicine through a pump.

38. The method of claim 20, wherein the step of connecting the medical devices to the patient further comprises the following step:
Recording and verifying the physical arrangement of the system.

39. The method of claim 20, wherein the step of connecting the medical devices to the patient further comprises the following step:
Entering the connection between the medical devices and between medical devices and the patient as a schematic diagram on the LCC.

40. The method of claim 41, further comprising the following step:
Recording the schematic to the server as a part of the patient medical record.

41. The method of claim 41, further comprising the following step:
Including the configuration of medical devices with respect to the patient.

42. The method of claim 41, further comprising the following step:
Providing a schematic diagram of most commonly used configurations such as use of a single, double, or triple drip IV, combination of IV with vital sign monitoring available to the medical staff on LCC in a drop-down menu.

43. The method of claim 20, wherein the step of connecting the medical devices to the patient further comprises the following step:
Providing a software package on the LCC where more complex arrangement of medical equipment connected to the patient can be recorded and saved as a part of the patient's medical record.

44. The method of claim 20, wherein the step of connecting the medical devices to the patient further comprises the following step:
Exchanging information between the LCC and the medical devices over a uniform wireless protocol determined by the WTU connected to them.

45. The method of claim 20, wherein the step of connecting the medical devices to the patient further comprises the following step:
Communicating from the WTU with the medical devices using different communication protocols.

46. The method of claim 45, wherein the step of communicating from the WTU with the medical devices using different communication protocols is performed using an application level protocol which defines a uniform method for exchanging data between the WTU and the LCC.

47. The method of claim 45, wherein the step of communicating from the WTU with the medical devices using different communication protocols is performed using an application level protocol which defines a uniform method for exchanging data between the WTU and the LCC which meets the specification of Sensitron Personalization Protocol (SSP) as described in detail in U.S. patent application Ser. No. 10/850,527 assigned to the Applicant herein having filing date May 19, 2004.

48. The method of claim 20, wherein the step of transferring communication and control of the medical devices completely from the LCC directly through an access point to the wireless data network further comprises the following step:
Resolving any error condition generated during transmission by the server and the WTU using different protocols as required.

49. The method of claim 20, wherein the step of transferring communication and control of the medical devices completely from the LCC directly through an access point to the wireless data network further comprises the following step:
Upon a break in the network availability, the server informing the medical staff using email, audio-visual indicators or any other suitable means such as a synthesized spoken message that the network connection to a specific patient monitors has become unavailable, and certain corrective actions need to be taken to remedy the problem.

50. The method of claim 20, wherein the step of transferring communication and control of the medical devices completely from the LCC directly through an access point to the wireless data network further comprises the following step:
Programming the WTU to record data during network outage and store the data locally while the network connection is unavailable.

51. The method of claim 20, wherein the step of transferring communication and control of the medical devices completely from the LCC directly through an access point to the wireless data network further comprises the following step:
Programming the WTU to present an audiovisual display to represent a lost network connection.

52. The method of claim 20, wherein the step of transferring communication and control of the medical devices completely from the LCC directly through an access point to the wireless data network further comprises the following step:
Storing any error messages generated by the medical devices by the WTU, optionally giving priority over data storage.

53. The method of claim 20, wherein the step of transferring communication and control of the medical devices completely from the LCC directly through an access point to the wireless data network further comprises the following step:
Delivering any error messages to the server when the connection is re-established.

54. The method of claim 20, wherein the step of identifying the care provider to the system by connecting with the server using a browser in a local communication controller using a secure sign-on procedure further includes the use of a password.

55. The method of claim 20, wherein the step of identifying the care provider to the system by connecting with the server using a browser in a local communication controller using a secure sign-on procedure further includes the use of an appropriate biometric measurement selected from the group consisting of fingerprint analysis, retinal scan and voice recognition.

56. The method of claim 20, wherein the step of authenticating all the medical devices in the bedside area network further comprises the following step:
Downloading parameters into the WTU such as the name and addresses of the network and the server where the WTU is required to connect upon powering up.

57. The method of claim 20, wherein the step of authenticating all the medical devices in the bedside area network further comprises the following step:
Authenticating a secret key downloaded in the WTU from the server for authenticating itself.

58. The method of claim 20, wherein the step of authenticating all the medical devices in the bedside area network further comprises the following step:
Building a table in the server containing any one or more of the following: the patient ID, the MAC address of the WTU, its AMI tag or code, and its secret key or SSID.

59. The method of claim 20, wherein the step of authenticating all the medical devices in the bedside area network further comprises the following step:
Using identifiers, or AMI, including a serial number of a WTU which is read by the medical staff and entered manually, entered as a picture taken from a miniature camera, read using a bar-code reader, or using a RFID tag reader.

60. The method of claim 20, wherein the step of authenticating all the medical devices in the bedside area network further comprises the following step:
Encrypting the MAC address of the WTU and transforming it into a barcode or another identifier which is also attached to the outside of WTU.

61. The method of claim 20, wherein the step of authenticating all the medical devices in the bedside area network further comprises the following step:
providing an audio or visual indication of all the WTUs at the bedside identified to have connected with the server.

62. The method of claim 20 wherein only the WTUs being used in the patient care at a specific bedside are identified at the server.

63. The method of claim 62 wherein only the WTUs identified at the server are assigned a BAN index.

64. The method of claim 20 in which the patient ID which is stored in the WTU is used to identify the medical devices needed for patient care and assign them to a BAN.

65. A method for provisioning and control of IV pumps using wireless data communication, the method comprising the following steps:
Providing one or more IV pumps equipped with a WTU capable of transmitting and receiving data over a wireless network having a data server;
Identifying a care provider to the system by connecting with the server using a browser in a local communication controller using a secure sign-on procedure;
Verifying the identity of the patient according to data from the server;
Identifying the details of the physician's instructions and the one or more IV pumps required to fulfill the physician's instructions at the bedside of the patient;
Authenticating all the one or more IV pumps in the bedside area network by the server with the LCC;
Connecting all the one or more IV pumps arranged at the patient bedside to the patient according to the physician's instructions;
Initiating the one or more IV pumps either manually by the care provider or upon a command from the LCC to the one or more WTUs connected to the corresponding medical devices, while recording the process data to the LCC;
Transferring communication and control of the one or more IV pumps completely from the LCC directly through an access point to the wireless data network; and
Delivering process or alarm data from the server to any client device connected to the wireless network, the client devices selected from the group consisting of a desktop notebook, a wireless notebook or a PDA, a pager capable of receiving text or numerical messages, or a cellular phone capable of receiving textual messages.

66. A protocol for establishing a BAN, such as for using medical instruments communicating using a wireless data network, the protocol comprising the following steps:
Attaching an AMI tag to each of one or more WTUs;
Programming each of the WTUs with the server, network and AMI identifier;
Attaching each of the WTUs to a different piece of medical equipment or monitoring device to be used in the treatment of the patient;
Powering up each piece of medical equipment or monitoring device at the bedside of the patient;
Initiating and completing secure sign-on and registration of an LCC with the server;
Searching by each of the WTUs on each piece of medical equipment or monitoring device for a wireless network;
Completing secure sign-on by each of the WTUs;
Logging the AMI identifiers and IP addresses of each of the WTUs in a database;
Providing visual or audio confirmation of completing secure sign-on or logging AMI identifiers and IP addresses of each of the WTUs in a database;
Debugging the lost network connection if registration of any one of the WTUs in incomplete;
Sending a list of all registered WTUs from the server to the LCC when registration of all the WTUs is complete;
Prompting the medical staff for AMI entry for all registered WTUs;
Confirming WTU association with the BAN by providing audio or visual indicator;
Assigning a name to the BAN by the server once all of the WTUs at the bedside have been identified;
Entering the patient's ID;
Programming the medical devices by the medical staff according to instructions provided by medical physician or other professionals;
Connecting the medical devices to the patient;
Recording the process configuration and operating parameters on the LCC; and
Providing medical treatment or monitoring with the medical devices of the BAN.

67. A method for moving a patient from one location to another, the method comprising the following steps:
Logging into the system with a transient LCC;
Obtaining all of the IP addresses of all the WTUs used in the first BAN;
Communicating data to and from the server over a network accessing the network at one or more access points,
Storing data locally by the WTUs when the connection to the network is lost during transit;
Forming a BSS with the WTUs as network nodes if connection with the network is unavailable after a predetermined period of time in order to allow the transient LCC to gather data from the WTUs as long as the connection to the network is interrupted and retrieval of the data from the LCC can be made at the destination;
Downloading stored data to the server after achieving a re-connection with the network; and
Continuing to monitor the medical device.
